(19) United States
(12) Patent Application Publication
(10) Pub. No.: US 2021/0244309 A1
(43) Pub. Date: Aug. 12, 2021

(54) METHOD OF CALIBRATING MAGNETIC PARTICLE IMAGING SYSTEM

(71) Applicants: ASELSAN ELEKTRONIK SANAYI VE TICARET ANONIM SIRKETI, Ankara (TR); IHSAN DOGRAMACI BILKENT UNIVERSITESI, Ankara (TR)

(72) Inventors: Can Baris TOP, Ankara (TR); Serhat ILBEY, Ankara (TR); Alper GUNGOR, Ankara (TR); Huseyin Emre GUVEN, Ankara (TR); Tolga CUKUR, Ankara (TR); Emine Ulku SARITAS CUKUR, Ankara (TR)

(73) Assignees: ASELSAN ELEKTRONIK SANAYI VE TICARET ANONIM SIRKETI, Ankara (TR); IHSAN DOGRAMACI BILKENT UNIVERSITESI, Ankara (TR)

(21) Appl. No.: 17/054,541

(22) PCT Filed: May 11, 2018
(86) PCT No.: PCT/TR2018/050225

§ 371 Date: Nov. 11, 2020

(51) Int. Cl. A61B 5/0515; A61B 5/00; G01R 33/12; G01R 35/00

(52) U.S. Cl. CPC A61B 5/0515; A61B 5/7257; A61B 2560/0223; G01R 35/005; G01R 33/1276

(57) ABSTRACT

A method of calibrating a magnetic particle imaging system including a magnetic field generator and a measurement device by proposing a coded calibration scene, wherein the coded calibration scene contains multiple nanoparticle samples distributed inside a volume of the coded calibration scene, larger than a field of view, wherein the coded calibration scene is moved linearly in one or more directions and/or rotated at one or more axes on the magnetic imaging system, and further, a mechanical system for moving the coded calibration scene.

### CROSS-REFERENCE TO THE RELATED APPLICATIONS

This application is the national stage entry of International Application No. PCT/TR2018/050225, filed on May 11, 2018, the entire contents of which are incorporated herein by reference.

### TECHNICAL FIELD

The present disclosure relates a method of calibrating magnetic particle imaging system by proposing a coded calibration scene that contains multiple nanoparticle samples distributed inside its volume, larger than the field of view on which the scene is moved linearly in one or more directions and/or rotated at one or more axes, and further, a mechanical system for moving the coded calibration scene.

### BACKGROUND

Magnetic nanoparticles can be used for various purposes in medicine such as angiography, stem cell tracking, imaging of cancerous cells and targeted drugs. Magnetic nanoparticles can be imaged non-invasively using the Magnetic Particle Imaging (MPI) method. Two different methods are used as standard for image reconstruction in the magnetic particle imaging method.

The first one is the system calibration method as stated in U.S. Pat. No. 8,355,771B2 numbered patent, in which a small volume nanoparticle sample is scanned mechanically at the desired system resolution steps in the field of view to obtain the calibration data of the system [1]. Images are generated using this calibration data (which is also called the system matrix). In the standard system calibration method, the calibration measurements last very long since the sample nanoparticle must be mechanically scanned and measured at every grid point in the field of view. The mechanical scanning time from one point to the other and acquisition of the measurement data takes about 1.3 sec [2]. For a small field of view having 30×30×30 grid points, the calibration time lasts 9.75 hours. In clinical practice, the calibration of a larger imaging volume may last for months. There is a need to calibrate the system frequently, since nanoparticle characteristics are known to vary from batch to batch and is also affected by the imaging sequence. For this reason, standard system calibration method cannot be practically adopted for systems with large field of view. In addition, since the nanoparticle to be scanned must be smaller than or equal to the voxel size of the image, the number of nanoparticles in the scanned sample is limited and the signal-to-noise ratio is small. A method to increase the signal-to-noise ratio is multiple data acquisition at the same position, and averaging. Therefore, the mechanical motion cannot be continuous, and the scanner should be stopped at every grid point, and moved to the next point after taking enough measurements to reach to the desired signal noise level. This limits the speed of the calibration measurements.

Recently, a calibration method has been proposed in an application no. US20150221103A1, in which the nanoparticle sample is scanned at random positions much fewer than the total number of voxels in the field of view. This is possible according to the compressed sensing theory [3] since the system matrix is sparse in certain transform domains (discrete Fourier, cosine, or Chebyshev). It has been shown that this method can reduce the number of scanned points by 80-90%. Instead of taking measurements from all of the voxels (N) in the field of view, system calibration can also be done by making reduced number of measurements at random M (<N) voxel positions using compressed sensing techniques. Since it is not possible to calculate how small M should be analytically, the M/N ratio should be chosen according to the image quality. Experimental images were obtained in the above mentioned reference. While image quality was acceptable for M/N=0.1, it was significantly degraded for lower M/N ratios. The calibration time can be reduced by a factor of 10 with this method but very long calibration times are still needed since the sample is mechanical scanned, i.e. a measurement area of 200×200×200 points will take longer than 10 days to measure.

The second reconstruction method is the X-space approach used in application numbered EP3143929A1. In this method, there is no calibration step; images are generated using the signal equation model for the magnetic particles imaging. Image reconstruction is done in the time domain by using the MPI signal equation. In this method, deviations from the ideal of MPI hardware are not taken into account and the resolution is lower than the system calibration method.

A von Gladiss et al. [2] discloses an electronic calibration method for accelerating the calibration procedure. The nanoparticle sample is placed in a separate calibration unit, which can generate homogeneous magnetic fields of any orientation imitating the magnetic fields that the nanoparticle sample would be exposed in the MPI system. Although this method provides faster calibration than the standard method, it requires the use of a separate calibration unit; the magnetic field distribution of the MPI system must be separately measured in the field of view; and the calibration unit measurements must be related to the MPI system measurements. Since magnetic field distribution measurements of the MPI system require mechanical scanning at each voxel in the field of view, as in the case of standard system calibration measurements, the advantage of electronic calibration is limited.

### SUMMARY

In the present invention, a large calibration apparatus, which will be referred as a coded calibration scene, is proposed for the calibration of an MPI system. Coded calibration scene includes nanoparticle samples at multiple positions. It is moved linearly in one or more directions, and/or rotated about one or more axes. During this movement, calibration measurement data are acquired at certain positions of the coded calibration scene. System matrix is generated using this measurement data with compressed sensing methods. The advantages that distinguish this method from other available methods are listed below:

In the state of the art, US patent no. US20150221103A1, a single nanoparticle sample is mechanically scanned for M voxels selected randomly or pseudo-randomly from the total number of voxels (N) in the field of view one by one for MPI system calibration. In the present invention, a calibration apparatus is proposed, which includes multiple nanoparticle samples and is larger than the field of view of the imaging system at least in one direction. Thus, the level of the received signal is much higher compared to the level of the signal received from a single nanoparticle sample. This allows the measurements to be made during continuous movement of the calibration scene, speeding up the calibration procedure substantially. In addition, the information content of the each measurement is increased as the nanoparticle samples at different positions are measured at the same time in a single measurement. Therefore, the system calibration matrix can be formed using fewer measurements. This provides an advantage for systems with large field of view.

In the method proposed by A von Gladiss et al. [2], a separate calibration unit is used for nanoparticle characterization. Therefore, the measurement of the magnetic field in the field of view of the MPI system is necessary. In the present invention, all the effects (magnetic field inhomogeneities, nanoparticle response) are taken into account in a single calibration scan.

### BRIEF DESCRIPTION OF THE DRAWINGS

FIG. 1 shows the cross section of the bore of a magnetic particle imaging setup, the non-homogeneous primary magnetic field with two zones and homogeneous secondary magnetic field, and the field of view.

FIG. 2 shows an entire field of view that is hypothetically divided into small voxels and a calibration setup using a sample containing the nanoparticles.

FIG. 3 shows a coded calibration scene with a plurality of nanoparticle samples distributed randomly or pseudo-randomly inside its volume.

FIG. 4 shows comparison of the standard compressed sensing method with the proposed method for the same noise level using a simulation model. Proposed method shows better image quality with smaller number of measurements (M).

FIGS. 5 and 6 show nanoparticle locations of a spherical calibration scene at 0 and 45 degree angles, respectively.

FIG. 7 shows a spherical calibration scene that rotates in one axis and slides in another axis.

FIG. 8 shows a calibration stage and a rotating mechanism rotate about different rotational axes.

FIGS. 9 and 10 show a spherical calibration scene and an external mechanism for linear and rotational movement of the spherical calibration scene in top and side views, respectively.

FIG. 11 shows a calibration scene including nanoparticle chambers connected to each other by thin channels and one or more points for being filled in or discharged.

FIG. 12 shows a spherical scene with rod shaped nanoparticle specimens.

FIG. 13 shows a calibration scene designed as a long rectangular prism making linear motion on a sliding belt.

FIGS. 14 and 15 show a cylindrical calibration scene and an external mechanism for linear and rotational movement of the scene from the top and the side, respectively.

FIG. 16 shows a cylindrical calibration scene with columnar cavities for nanoparticle samples.

FIG. 17 shows a cylindrical calibration scene with a thin tube in the form of a complex curve in three dimensions with an input and an output.

### PART REFERENCES

1. MPI system
2. Primary magnetic field
3. First zone of the primary magnetic field
4. Second zone of the primary magnetic field
5. Secondary magnetic field
6. Field of view
7. Voxel
8. Magnetic nanoparticle sample
9. Mechanical scanner
10. Coded calibration scene
11. Spherical calibration scene
12. Rotation center
13. Mechanism for translating and rotating a calibration scene around one axis
14. Mechanism for translating and rotating a calibration scene around two axes
15. Auxiliary mechanical system for translating and rotating a calibration scene
16. Railed slide
17. Rotation axis
18. Thin channel
19. Opening
20. Rod shaped nanoparticle sample
21. Rectangular prism calibration scene
22. Sliding belt
23. Optical reflector
24. Laser tracker
25. Cylindrical calibration scene
26. Columnar cavity
27. Input for filling the magnetic nanoparticles inside the calibration scene
28. Output for draining the magnetic nanoparticles inside the calibration scene
29. Thin tube

### DETAILED DESCRIPTION OF THE EMBODIMENTS

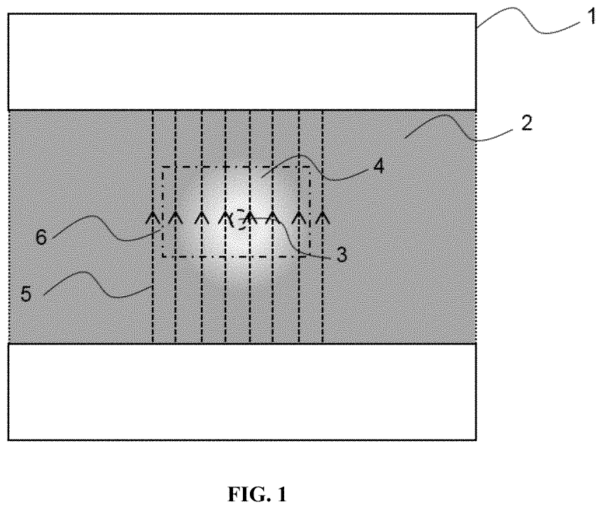

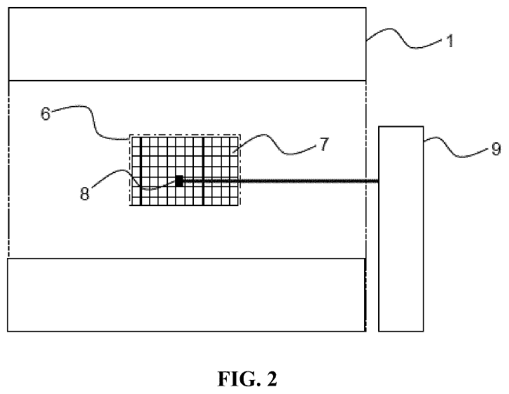

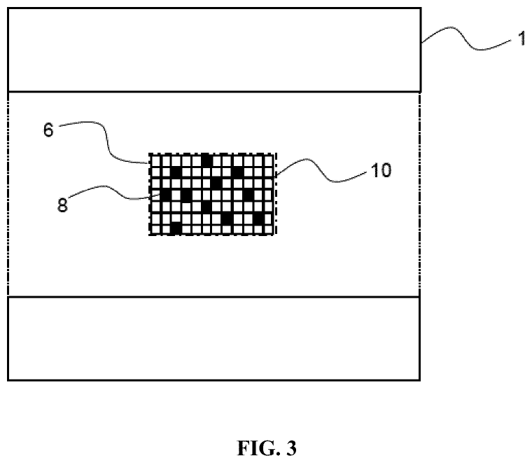

In an MPI system (1) that consists of a magnetic field generator and a measurement device as shown in FIG. 1, the distribution of magnetic nanoparticles is imaged using a non-homogeneous primary magnetic field (2) having two zones [4]. The first (3) of these two zones has a very low magnetic field intensity and is called the field free region (FFR). The magnetic nanoparticles in the FFR can be magnetized in the direction of a secondary external magnetic field (5). In the second zone (4), the magnetic field intensity is high and the magnetic nanoparticles in this region are in saturation. Therefore, they respond marginally to a secondary magnetic field (5). The secondary magnetic field (5) is applied to the entire field of view (6) as a time varying magnetic field. The time-dependent magnetization of the magnetic nanoparticles in the FFR is measured by the receiving coil(s). The amplitude of the measured signal is directly proportional to the number of nanoparticles in the FFR. The FFR is scanned electronically or mechanically throughout the field of view (6) to obtain the nanoparticle distribution in the field of view (6). Since the magnetic nanoparticles have a non-linear magnetization curve, the received signal from the particles in the FFR contains the harmonics of the frequency of the transmitted signal. The received signal properties depend on the properties of the nanoparticle (size, shape, material, etc.) and the nanoparticle environment (viscosity, temperature), and properties of the magnetic field of the imaging system. In MPI, best image quality is achieved with the image reconstruction method based on the system calibration method, which takes all these effects into account [5].

In the system calibration image reconstruction method, firstly the entire field of view (6) is hypothetically divided into small voxels (7). A system matrix is formed using a sample (8) filled with a magnetic nanoparticle having a size of a voxel (7). For this, the sample (8) containing the nanoparticles is scanned to every voxel position by means of a mechanical scanner (9). Secondary magnetic field signal is applied, and the nanoparticle signal received by the receiving coils is stored in a digital storage unit (e.g. hard disk). In practice, the measurement data are acquired multiple times at the same voxel point, and the signal to noise ratio is increased by averaging the measurements data. The measured signal from a single voxel is converted to the frequency domain using the Fourier transform, forming a column of the system matrix (A). The whole system matrix is generated by taking measurements at all voxel positions. This process is called the calibration step.

For imaging, measurement data are acquired by scanning the FFR inside the object, and the image is reconstructed using this measurement data and the system matrix. To this end, a linear equation set Ax=b is solved. In this equation set, A is the system matrix, b is the vector of measurements taken from the object, and x is the nanoparticle distribution inside the object. The major disadvantage of the system matrix calibration method is its long duration (about 1.3 seconds per voxel, multiplied by the number of voxels) [2]. In addition, since the sample size of the nanoparticle is very small, the signal level is low and it is necessary to increase the signal-to-noise ratio by taking multiple measurements. This prevents continuous mechanical scanning, leading to the prolongation of the measurement period.

The present invention proposes the use of coded calibration scenes (10) to solve the problems of the prior art. A coded calibration scene can be defined as an apparatus containing a plural number of nanoparticle samples, distributed inside its volume. This method has the following advantages: The signal level increases proportional to the number of particles used in the calibration scan, and the condition of the compressed sensing problem is increased [6]. As a result, calibration is possible with fewer number of measurements using compressed sensing algorithms such as greedy reconstruction algorithms, approximate message passing, optimization based techniques, etc. [3]. According to the compressed sensing theory, the correlation of calibration scenes with each other should be minimized. For this reason, nanoparticles can be distributed randomly or pseudo-randomly in each calibration scene.

An implementation of this method is as follows: the number of calibration scenes, M, to be measured is predetermined. For this, the simulation model of the imaging system can be used, or a number of calibration scenes are produced during the system tests of the produced imaging system; new scenes are measured until the image quality reaches a sufficient level from the clinical point of view. The measurement data are collected and recorded for M coded calibration scenes. Once these measurements have been taken, the system matrix, A, is reconstructed using the following optimization problem:

$${argmin }_{A} {‖ {DA}^{T} ‖}_{1} , subject to {‖ {PA}^{T} - {A}_{p} ‖}_{2} < {ɛ}_{p}$$

where P is the nanoparticle density matrix for the measured coded calibration scenes, D is the transformation matrix associated with a sparsifying transform such as discrete Fourier transform, discrete Chebyshev transform, discrete cosine transform, or any other transform where the vector can be represented with fewer coefficients than its original domain; Aᵥ is the measurement matrix converted to Fourier space for each measurement position; εᵥ represents a constant related to the error caused by the system noise. Different algorithms in the literature can be used to solve the above optimization problem (e.g. Fast Iterative Shrinkage Thresholding Algorithm (FISTA), Alternating Direction Method of Multipliers (ADMM) [7]). Moreover, adding similar regularization functions or using the unconstrained form will not change the described benefits of the invention.

This method is compared with the standard compressed sensing method for the same noise level using a simulation model as revealed in FIG. 4. An object with N=3200 pixels was imaged both by using the standard compressed sensing calibration method with M=2560 calibration points and M=320 coded calibration scenes. The resultant image quality was poor for the standard compressed sensing method, while a high quality image was obtained with the coded calibration scenes.

In an embodiment, random points expressed by P can be selected from a domain that can be quickly transformed, such as the Hadamard matrix, in order to shorten the solution time of the problem given in the inequality. In this case, the P matrix can be expressed as a masked unitary transformation. It has previously been shown that the optimization problem can be solved efficiently in situations involving a masked unitary transformed space [8]. By this way, the problem of solution time can be further decreased.

In practice, the time for switching between the coded calibration scenes would be much longer than measurement time of a single coded calibration scene. Therefore, the total calibration duration would be determined by the total number of coded calibration scenes used and the time required for changing (replacement) of the coded calibration scenes. In order to mitigate this problem, a calibration scene that is larger than the field of view at least in one direction is proposed with the present invention. Instead of changing the calibration scenes one by one, the scene is moved linearly in one or more directions and/or rotated at one or more center points. Calibration measurements are taken at certain positions during continuous movement. The nanoparticle distribution in the imaging field of view changes as a function of time. Therefore, at different time instants, a different part of the calibration scene is present in the field of view. In a preferred embodiment, the measurement is taken during continuous motion of the calibration scene. This is possible when the signal noise ratio is high as a result of large number of nanoparticles used in the calibration scene. Consequently, there is no need to repeat and average the measurements. In this way, it is possible to shorten the measurement time substantially. As a result, it is possible to calibrate the system frequently to obtain a high image quality.

The locations of the nanoparticle samples in the calibration scene must be known precisely. The calibration scene can be produced with high-precision production methods and/or can be measured after production with high resolution imaging methods such as X-ray imaging.

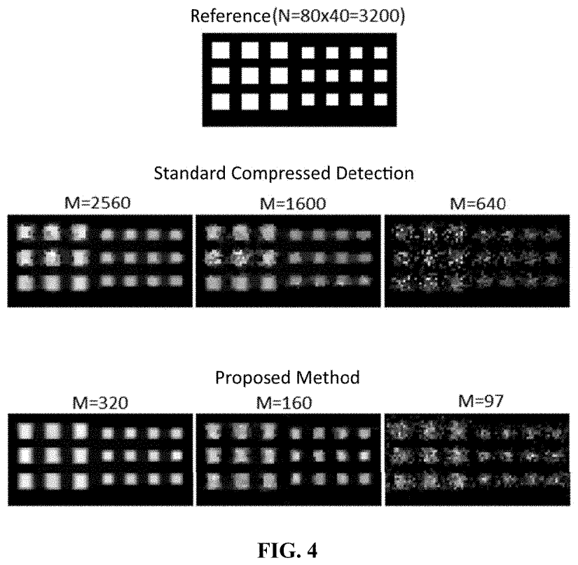

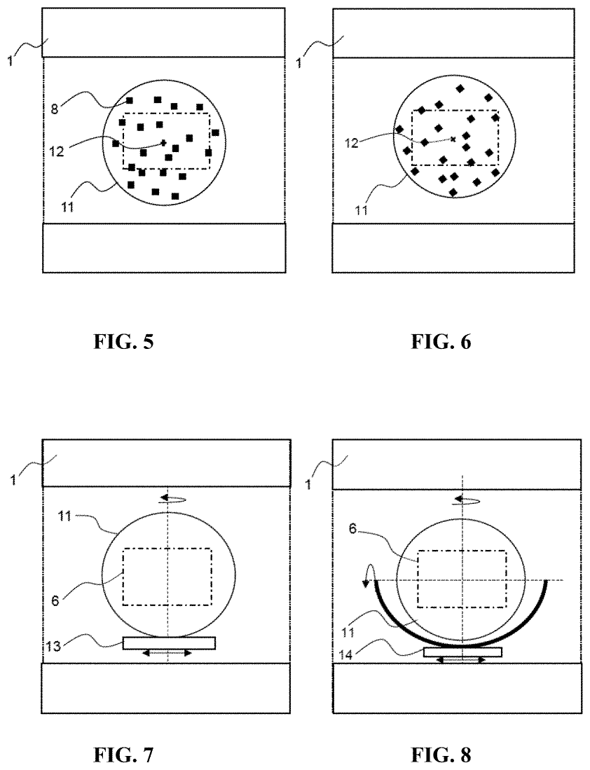

The calibration scene can be moved linearly and/or circularly. In an example embodiment, a spherical calibration scene is rotated about one axis and measurements are taken at K degrees intervals. The position of the nanoparticles samples (8) in the calibration scene change as a function of rotation angle. For example, the nanoparticle locations of a spherical calibration scene (11) at 0 and 45 degree angles are given in FIGS. 5 and 6, respectively. For each rotation angle, the nanoparticle's new position in the field of view grid, and the nanoparticle density at each grid point in the new location are calculated. The error in this calculation depends on the accuracy of the rotation measurement of the rotation mechanism. If this accuracy is not sufficient, the new position can be precisely measured using a position tracker with high sensitivity, such as a laser tracker or a device of similar purpose. In order to obtain the system matrix with high accuracy, the process can be repeated at a number (L) of different rotation centers (12) to increase the amount of measurement data. The total number of measurements is then M=(360/K)*L. Once these measurements have been taken, the system matrix is reconstructed by solving the optimization problem given in above stated inequality. In the inequality, P is the matrix containing the nanoparticle density distribution in the field of view at each measurement position.

A mechanism for translating and rotating a calibration scene around one axis (13) can be used to move and/or rotate the calibration scene. The mechanism (13) required to rotate the calibration scene can be designed as an integrated unit or as an external unit to the MPI system (1). An example embodiment is shown in FIG. 7. Here, the spherical calibration scene (11) rotates in one axis and slides in another axis. In this way, it is possible to measure the calibration scene at different rotation centers with respect to the field of view center and increase the diversity of the calibration scene measurements. The linear sliding motion as well as the rotation motion can be continuous during the calibration reducing the calibration time compared to stepped motion.

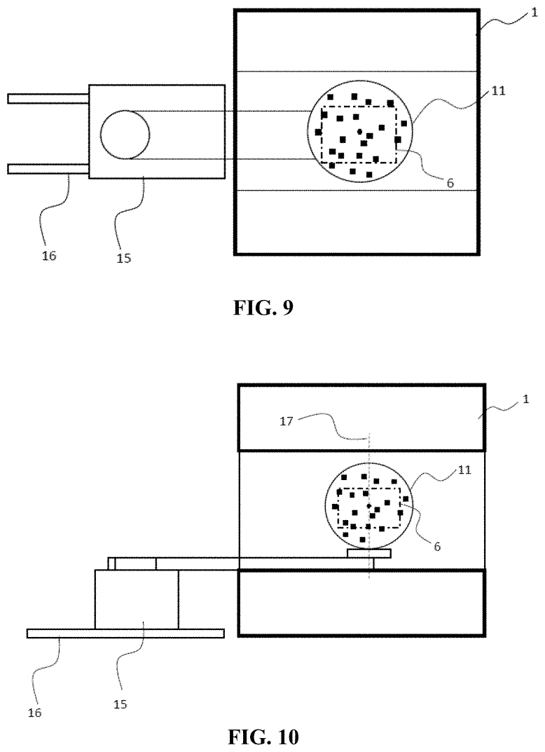

A mechanism for translating and rotating a calibration scene around two axes (14) can also be designed to rotate about different rotational axes as shown in FIG. 8. In this case, the conditioning of the autocorrelation of the P matrix can be improved, which is helpful for the solution of the optimization problem. In the implementation of the method, the calibration scene and the rotating mechanism can also be designed as an external unit according to the MPI system's mechanical requirements. Such an implementation is shown in FIG. 9 and FIG. 10. In FIG. 9, a spherical calibration scene (11) is shown in top view, which makes a linear sliding movement on a railed slide (16) and a rotational movement about a rotation axis (17) by means of a reel system. FIG. 10 shows the side view of this calibration system. An auxiliary mechanical system for translating and rotating a calibration scene (15) includes the necessary equipment (motor, encoder, motion transfer elements, and computerized control interface) to perform linear and rotational movements of the calibration scene. In a preferred embodiment the mechanical system includes a control unit, which communicates with the MPI system (1) to perform the calibration procedure using the calibration scenes. To this end, the control unit receives the required position of the calibration scene from the MPI system by electronical means, moves the calibration scene to the required position, outputs the position information of the calibration scene obtained from the encoders in the mechanical system and/or tracking device that measure the position of the calibration scene.

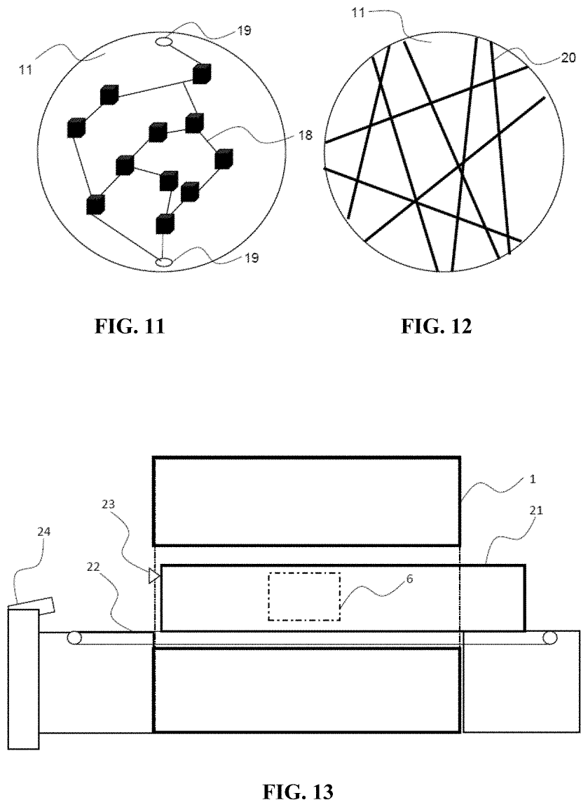

Calibration scenes should allow for rapid filling (and emptying) of different nanoparticles. In an embodiment, a three dimensional coded calibration scene can be formed by a plurality of mechanically separable layers allowing the nanoparticle samples to be changed. In another embodiment, a single layer calibration scene can be used for calibration in two-dimensions. It can be mechanically scanned in the third dimension to calibrate a three dimensional field of view. FIG. 11 shows another embodiment; a calibration scene including nanoparticle chambers connected to each other by thin channels (18), and openings (19) for filling or draining the magnetic nanoparticles inside the calibration scene. The calibration scene is a hollow structure with one or more openings for filling or emptying the structure with nanoparticles.

The nanoparticle samples present in the calibration scenes do not have to fit into a single voxel (10). Scenes can include nanoparticle samples of different sizes and shapes. For example, a nanoparticle sample can be of any shape such as spherical, elliptical or rectangular prism, and cover many voxels. In an embodiment, a spherical scene is considered, with rod shaped nanoparticle samples (20) as shown in FIG. 12. The rods can be easily taken out and inserted into the scene. The calibration scene can be produced in any arbitrary shape such as sphere, cylinder, cube, rectangular prism.

In another embodiment shown in FIG. 13, the calibration scene (21) designed as a long rectangular prism makes only linear motion on a sliding belt (22). Calibration measurements are made at certain positions through the field of view. FIG. 13 also shows an optical reflector (23) and a laser tracker (24) to ensure precise measurement of position during movement. One or more reflectors can be attached to the calibration scene for tracking its movement.

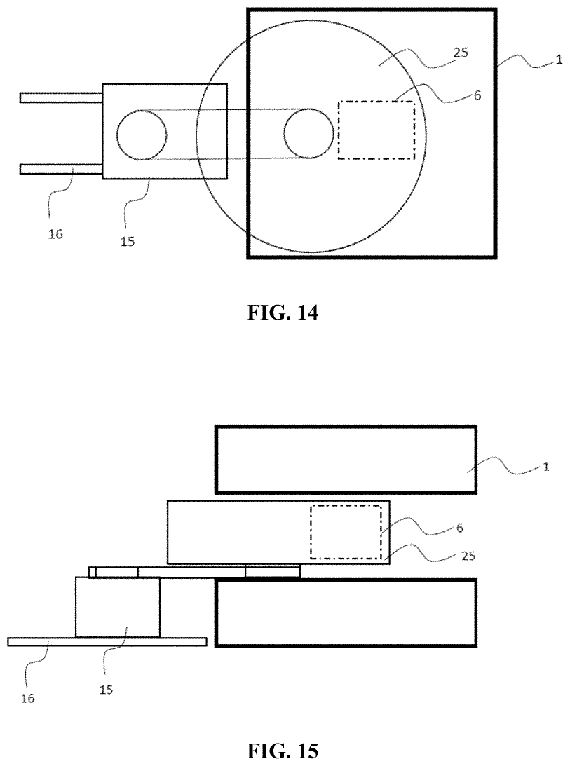

In the embodiments shown in FIGS. 14 and 15, a cylindrical calibration scene (25) is employed. Calibration can be performed at fewer number of rotations than that required by the calibration scene given in FIGS. 9 and 10, since the volume of the calibration scene is wider. However, such a calibration scene requires a large opening, which may be suitable for open bore MPI systems.

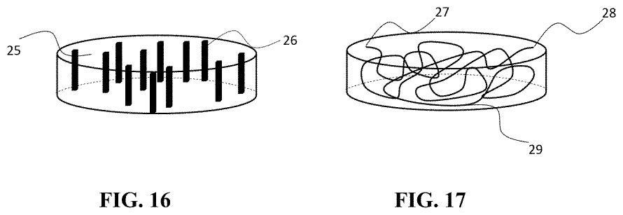

FIG. 16 shows an embodiment including columnar cavities (26) for nanoparticle samples that can be filled and emptied quickly.

FIG. 17 shows an embodiment which includes a thin tube (29) in the form of a complex curve in three dimensions with single input for filling (27) and an output for draining (28) the magnetic nanoparticles inside the calibration scene. The calibration scene may include a single or plural number of tubes of arbitrary path traversing the calibration scene for filling or emptying the tubes with nanoparticles.

### REFERENCES

[1] Weizenecker J, Gleich B, Rahmer J, Dahnke H, Borgert J (2009). Three-dimensional real-time in vivo magnetic particle imaging, Phys Med Biol. 2009; 54: L1-L10.
[2] A. von Gladiss, M. Graeser, P. Szwargulski, T. Knopp and T. M. Buzug. Hybrid system calibration for multidimensional magnetic particle imaging. Phys. Med. Biol., vol. 62, no. 9, pp. 3392, 2017.
[3] Compressed Sensing Theory and Applications, Ed. By Y. C. Eldar, G. Kutyniok, Cambridge University Press, New York, 2012.
[4] B. Gleich and J. Weizenecker. Tomographic imaging using the nonlinear response of magnetic particles. Nature, 435(7046):1217-1217, 2005. doi: 10.1038/nature03808.
[5] T. Knopp, J. Rahmer, T. F. Sattel, S. Biederer, J. Weizenecker, B. Gleich, J. Borgert, and T. M. Buzug. Weighted iterative reconstruction for magnetic particle imaging. Phys. Med. Biol., vol. 55, no. 6, pp. 1577-1589, 2010. doi:10.1088/0031-9155/55/6/003.
[6] G. R. Arce, D. J. Brady, L. Carin, H. Arguello, and D. S. Kittle, “Compressive Coded Aperture Spectral Imaging,” IEEE Signal Processing Magazine, vol. 31, no. 1, pp. 105-115, 2014.
[7] S. Ilbey et al., “Comparison of system-matrix-based and projection-based reconstructions for field free line magnetic particle imaging,” International Journal on Magnetic Particle Imaging, vol. 3, no. 1, 2017.
[8] H. E Güven, A. Güngor, and M. Cetin, “An Augmented Lagrangian Method for Complex-Valued Compressed SAR Imaging,” IEEE Trans. Comput. Imaging, 2(3):235-250, 2016.

What is claimed is:

1. A calibration method for a magnetic particle imaging system to perform a magnetic particle imaging of a field of view comprising the steps of;
moving a calibration scene linearly in one or more directions and/or rotating about one or more axes by a mechanical system;
scanning a field free region in the field of view and acquiring calibration measurement data at a plurality of positions of the calibration scene; and
reconstructing a system matrix with compressed sensing methods by using the calibration measurement data and position information of the calibration scene during data acquisition.

2. The calibration method for the magnetic particle imaging system according to claim 1, comprising the step of reconstructing the system matrix using the following optimization problem subject to an inequality:
$${argmin }_{A} {‖ {DA}^{T} ‖}_{1} , subject to {‖ {PA}^{T} - {A}_{p} ‖}_{2} < {ɛ}_{p} ;$$
where P is a nanoparticle density distribution in the field of view at each measurement position; D is a matrix associated with a sparsifying transform for A, the system matrix; Aₚ is a measurement matrix converted to a Fourier space for the each measurement position of the calibration scene; εₚ represents a constant related to an error caused by a system noise.

3. The calibration method of claim 1, wherein the calibration scene is moved or rotated continuously.

4. The calibration method of claim 1, wherein the calibration scene comprises a plurality of nanoparticle samples.

5. The calibration method of claim 4, wherein the plurality of nanoparticle samples in the calibration scene are distributed randomly or pseudo-randomly.

6. The calibration method of claim 4, wherein the plurality of nanoparticle samples in the calibration scene are connectively distributed for filling and emptying from two ends of the calibration scene.

7. The calibration method of claim 1, wherein a position of the calibration scene is continuously monitored using a tracking device to measure the position of the calibration scene during the data acquisition.

8. A calibration apparatus for a magnetic particle imaging system, comprising:
a calibration scene with distributed nanoparticle samples inside a volume of the calibration scene, larger than a field of view of the magnetic particle imaging system,
a mechanical system performing linear movements in one or more directions and/or rotational movements around one or more axes of the calibration scene,
wherein the calibration scene comprises at least one tube of an arbitrary path traversing the calibration scene, for filling and emptying the tube with nanoparticles.

9. The calibration apparatus of claim 8, wherein an outer geometry of the calibration scene is a rectangular prism, a cylinder, a sphere or an arbitrary shape.

10. The calibration apparatus of claim 8, wherein one or more reflectors are attached to the calibration scene for tracking a movement of the calibration scene.

11. (canceled)

12. (canceled)

13. The calibration apparatus of claim 8, wherein the calibration scene is a hollow structure with one or more openings for filling or emptying the hollow structure with the nanoparticles.

14. The calibration apparatus of claim 8, wherein a position of the calibration scene is tracked by a tracking device.

15. The calibration apparatus of claim 8, wherein the mechanical system comprises a control unit, wherein the control unit communicates with the MPI system to carry out operations for a calibration method, wherein the calibration method comprising:
moving a calibration scene linearly in one or more directions and/or rotating about one or more axes by a mechanical system;
scanning a field free region in the field of view and acquiring calibration measurement data at a plurality of positions of the calibration scene; and
reconstructing a system matrix with compressed sensing methods by using the calibration measurement data and position information of the calibration scene during data acquisition.
